(19) United States
(12) Patent Application Publication
(10) Pub. No.: US 2022/0409269 A1
(43) Pub. Date: Dec. 29, 2022

(54) NASAL NERVE DENERVATION INSTRUMENT WITH DENERVATION CONFIRMATION

(71) Applicant: Acclarent, Inc., Irvine, CA (US)

(72) Inventors: Jetmir Palushi, Irvine, CA (US); Alison D. Wright, Newport Beach, CA (US); Shubhayu Basu, Anaheim, CA (US); Jeffrey D. Hopkins, Austin, TX (US)

(21) Appl. No.: 17/752,001

(22) Filed: May 24, 2022

Related U.S. Application Data
(60) Provisional application No. 63/214,372, filed on Jun. 24, 2021.

(51) Int. Cl. A61B 18/14

(52) U.S. Cl. CPC A61B 18/1485; A61B 2018/00434

(57) ABSTRACT

An apparatus includes a shaft assembly, first and second electrode assemblies, and a controller. The shaft assembly is configured to fit in a nasal cavity of a patient. The first and second electrode assemblies are at the distal end of the shaft assembly. The second electrode assembly includes a stimulus electrode and a sensing electrode. The stimulus and sensing electrodes are positioned on opposing lateral sides in relation to the longitudinal axis of the shaft assembly. The controller is operable to generate an electrical signal to perform one or both of tissue ablation or denervation of a targeted nerve via the first electrode assembly, generate an electrical stimulus signal to stimulate the targeted nerve via the stimulus electrode of the second electrode assembly, and process a response signal received from the targeted nerve via the sensing electrode of the second electrode assembly.

### PRIORITY

This application claims priority to U.S. Provisional Pat. App. No. 63/214,372, entitled “Nasal Nerve Denervation Instrument with Denervation Confirmation,” filed Jun. 24, 2021, the disclosure of which is incorporated by reference herein, in its entirety.

### BACKGROUND

Rhinitis is a medical condition that presents as irritation and inflammation of the mucous membrane within the nasal cavity. The inflammation results in the generation of excessive amounts of mucus, which can cause runny nose, nasal congestion, sneezing, and/or post-nasal drip. Allergenic rhinitis is an allergic reaction to environmental factors such as airborne allergens, while non-allergenic (or “vasomotor”) rhinitis is a chronic condition that presents independently of environmental factors. Conventional treatments for rhinitis include antihistamines, topical or systemic corticosteroids, and topical anticholinergics, for example.

For cases of intractable rhinitis in which the symptoms are severe and persistent, an additional treatment option is the surgical removal of a portion of the vidian (or “pterygoid”) nerve—a procedure known as vidian neurectomy. The theoretical basis for vidian neurectomy is that rhinitis is caused by an imbalance between parasympathetic and sympathetic innervation of the nasal cavity, and the resultant over stimulation of mucous glands of the mucous membrane. Vidian neurectomy aims to disrupt this imbalance and reduce nasal mucus secretions via surgical treatment of the vidian nerve. However, in some instances, vidian neurectomy can cause collateral damage to the lacrimal gland, which is innervated by the vidian nerve. Such damage to the lacrimal gland may result in long-term health complications for the patient, such as chronic dry eye. Posterior nasal neurectomy, or surgical removal of a portion of the posterior nasal nerves, may be an effective alternative to vidian neurectomy for treating intractable rhinitis.

FIG. 1 depicts a left sagittal view of a portion of a patient's head, showing the nasal cavity (10), the frontal sinus (12), the sphenoid sinus (14), and the sphenoid bone (16). The nasal cavity (10) is bounded laterally by the nasal wall (18), which includes an inferior turbinate (20), a middle turbinate (22), and a superior turbinate (24). The vidian nerve (32) resides within the vidian (or “pterygoid”) canal (30), which is defined in part by the sphenoid bone (16) and is located posterior to the sphenoid sinus (14), approximately in alignment with the middle turbinate (22). The vidian nerve (32) is formed at its posterior end by the junction of the greater petrosal nerve (34) and the deep petrosal nerve (36); and joins at its anterior end with the pterygopalatine ganglion (38), which is responsible for regulating blood flow to the nasal mucosa. The posterior nasal nerves (40) join with the pterygopalatine ganglion (38) and extend through the region surrounding the inferior turbinate (20).

While instruments and methods for performing vidian neurectomies, posterior nasal neurectomies, and turbinate reductions are known, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts a left sagittal view of a portion of a patient's head, showing details of certain paranasal sinuses and nerves, including the vidian nerve and the posterior nasal nerve;

FIG. 2 depicts a perspective view of an example of an instrument that may be used to perform an ablation procedure in a nasal cavity, with a pair of electrodes in a proximal retracted position relative to a shaft assembly of the instrument;

FIG. 3A depicts a perspective view of a distal portion of the shaft assembly of the instrument of FIG. 2, with a pair of needle electrodes in the proximal retracted position relative to the shaft assembly;

FIG. 3B depicts a perspective view of the distal portion of the shaft assembly of the instrument of FIG. 2, with the pair of needle electrodes in a distal extended position relative to the shaft assembly;

FIG. 4 depicts a perspective view of the distal portion of a variation of the shaft assembly of the instrument of FIG. 2;

FIG. 5 depicts a graph of a high frequency signal with a generally sinusoidal waveform;

FIG. 6 depicts a graph of a low frequency signal with a generally square waveform; and

FIG. 7 depicts a graph of a modulated signal combining the waveforms of the signals of FIGS. 5 and 6.

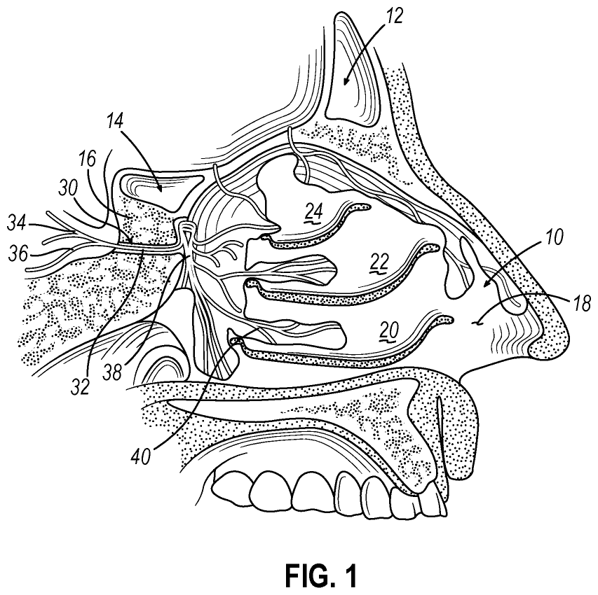

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for purposes of describing examples only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Examples of Shallow, Deep, and Volumetric Ablation

In some clinical scenarios, it may be desirable to apply electrical energy ((e.g., radiofrequency (AC type) or pulsed field (DC type) energy) to tissue to ablate the tissue, to provide reversible electroporation of the tissue, to provide irreversible electroporation of the tissue, or to otherwise treat the tissue. This may include contacting a surface of tissue with one or more electrodes, then activating the one or more electrodes to apply the electrical energy to the tissue. In cases where one electrode is used, a ground pad may be placed in contact with the skin of the patient, and the one electrode that contacts the targeted tissue surface may apply monopolar electrical energy to the targeted tissue surface. In cases where two or more electrodes are used, the two or more electrodes may be placed in contact with the targeted tissue surface and may be activated to apply bipolar electrical energy to the targeted tissue surface. Various suitable electrode arrangements may be used to provide electroporation. To the extent that the terms “ablation,” “ablate,” and variants thereof are used herein, such terms should be read as also including electroporation (reversible and irreversible), such that the inventors contemplate that all of the following teachings relating to ablation may also be applied in the context of electroporation.

Tissue treating electrodes may also come in the form of needles that penetrate tissue and are activated to apply electrical energy once the needles are inserted in tissue. Unlike tissue surface contacting tissue treatment electrodes, needle electrodes may facilitate ablation and/or other effects far past the surface of the tissue. In some cases, needle electrodes may avoid ablating or otherwise treating the tissue surface despite the penetration of the needle electrodes through the tissue surface, where only sub-surface tissue is ablated or otherwise treated.

In the context of some ear, nose, and throat (ENT) ablation procedures, it may be desirable to provide a relatively shallow RF ablation or other tissue treatment, such that only tissue surface contacting electrodes are used. In some other ENT scenarios, it may be desirable to provide a relatively deep RF ablation other tissue treatment, such that tissue penetrating needle electrodes are used. In still other ENT scenarios, it may be desirable to provide a combination of shallow ablation and deep ablation, thereby resulting in a volumetric ablation, through the combined use of tissue surface contacting electrodes and tissue penetrating needle electrodes.

In view of the foregoing, it may be desirable to provide an ablation instrument that is operable to perform relatively shallow ablation or other tissue treatment, relatively deep ablation or other tissue treatment, or volumetric ablation (i.e., combining shallow and deep ablation) or other tissue treatment, without requiring the use of more than one instrument. In other words, it may be desirable to provide a single instrument that is operable to transition between a shallow ablation/treatment modality, a deep ablation/treatment modality, and a volumetric ablation/treatment modality, subject the selection of the instrument operator. The following provides several examples of instruments that enable selectability between these modalities. While these examples are described in the context of ENT procedures, the instruments described below may be used in other procedures in other regions of a patient's anatomy as will be apparent to those skilled in the art in view of the teachings herein.

### II. Example of Instrument with Arcuate Electrodes, Needle Electrodes, and Camera Assembly

A. Overview

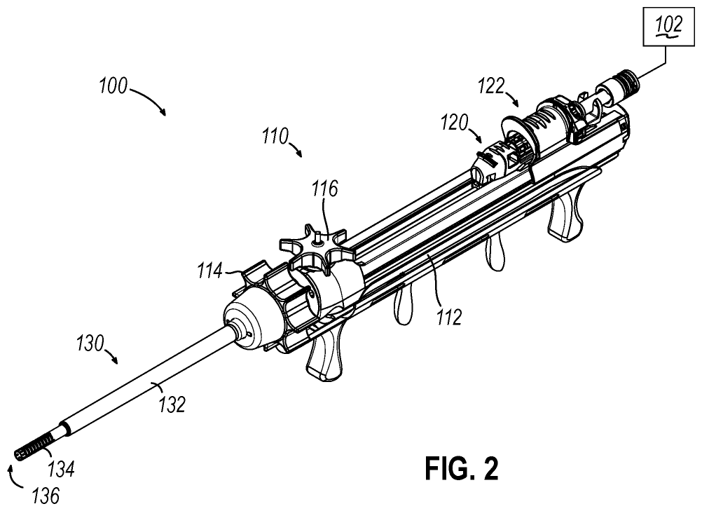

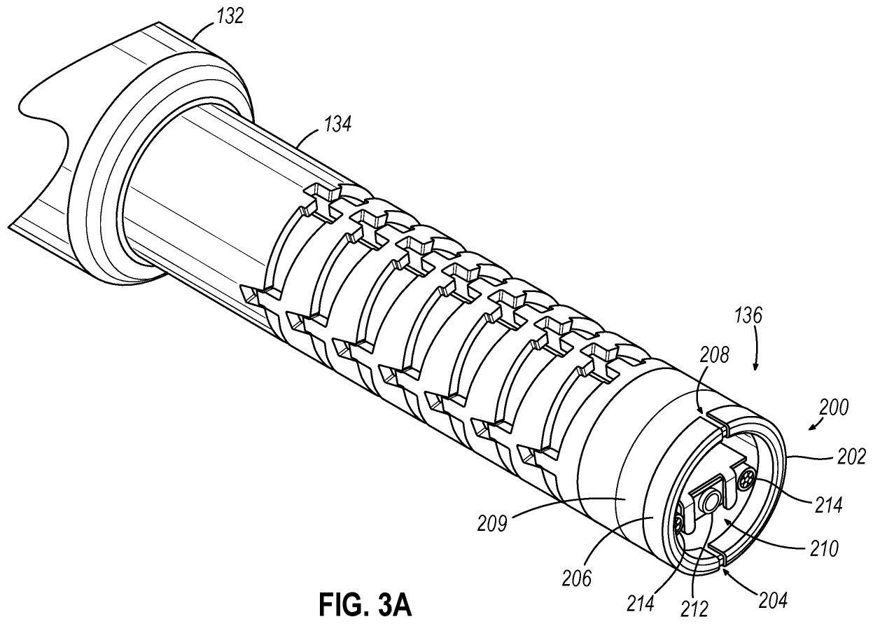

FIGS. 2-3B show an example of an instrument (100) that may be used to deliver electrical energy to tissue. For instance, instrument (100) may be used to ablate a nerve (e.g., the posterior nasal neve (40)); ablate a turbinate (e.g., any of turbinates (20, 22, 24)); or ablate, electroporate, or apply resistive heating to any other kind of anatomical structure in the head of a patient. Instrument (100) of this example includes a handle assembly (110), a shaft assembly (130), and an end effector (200) with electrodes (202, 206, 222, 226). Instrument (100) is coupled with an electrical generator (102), which is operable to generate electrosurgical energy for delivery to tissue via electrodes (202, 206, 222, 226) as will be described in greater detail below. Generator (102) may be incorporated into a controller that provides other functionality, including but not limited to position sensor signal processing, nerve stimulus signal processing, etc.

Handle assembly (110) of this example includes a body (112), a first slider (120), and a second slider (122). Body (112) is sized and configured to be grasped and operated by a single hand of an operator, such as via a power grip, a pencil grip, or any other suitable kind of grip. Each slider (120, 122) is operable to translate longitudinally relative to body (112). Sliders (120, 122) are operable to translate independently relative to each other in some versions. Slider (120) is coupled with a camera assembly (210) and is thus operable to translate camera assembly (210) longitudinally relative to shaft assembly (130). In some variations, camera assembly (210) is longitudinally fixed relative to shaft assembly (130), such that slider (120) may be omitted (210). Alternatively, slider (120) may be operable to longitudinally translate any other suitable component(s). Slider (122) is coupled with needle electrodes (222, 226) and is thus operable to translate needle electrodes (222, 226) longitudinally as will be described in greater detail below. The transition from FIG. 3A to FIG. 3B shows needle electrodes (222, 226) being driven by slider (122) from a proximal position to a distal position.

Shaft assembly (130) of the present example includes a rigid portion (132), a flexible portion (134) distal to rigid portion (132), and an open distal end (136). A pull-wire (not shown) is coupled with flexible portion (134) and with a deflection control knob (116) of handle assembly (110). Deflection control knob (116) is rotatable relative to body (112), about an axis that is perpendicular to the longitudinal axis of shaft assembly (130), to selectively retract the pull-wire proximally. As the pull-wire is retracted proximally, flexible portion (134) bends and thereby deflects distal end (136) laterally away from the longitudinal axis of rigid portion (132). Deflection control knob (116), the pull-wire, and flexible portion (134) thus cooperate to impart steerability to shaft assembly (130). By way of example only, such steerability of shaft assembly (130) may be provided in accordance with at least some of the teachings of U.S. Pub. No. 2021/0361912, entitled “Shaft Deflection Control Assembly for ENT Guide Instrument,” published Nov. 25, 2021, the disclosure of which is incorporated by reference herein, in its entirety. Other versions may provide some other kind of user input feature to drive steering of flexible portion (134), instead of deflection control knob (116). In some alternative versions, deflection control knob (116) is omitted, and flexible portion (134) is malleable. In still other versions, the entire length of shaft assembly (130) is rigid.

Shaft assembly (130) is also rotatable relative to handle assembly (110), about the longitudinal axis of rigid portion (132). Such rotation may be driven via rotation control knob (114), which is rotatably coupled with body (112) of handle assembly (110). Alternatively, shaft assembly (130) may be rotated via some other form of user input; or may be non-rotatable relative to handle assembly (110). It should also be understood that the example of handle assembly (110) described herein is merely an illustrative example. Shaft assembly (130) may instead be coupled with any other suitable kind of handle assembly or other supporting body.

### B. Examples of Electrodes

As best seen in FIGS. 3A-3B, an end effector (200) at distal end (136) of shaft assembly (130) includes arcuate electrodes (202, 206), needle electrodes (222, 226), and camera assembly (210). Each arcuate electrode (202, 206) of this example is in the form of a distally-facing, arcuate conductive element (e.g., metal) that is fixedly secured relative to distal end (136) of shaft assembly (130) via a cuff (209). Arcuate electrodes (202, 206) of the present example are angularly separated from each other by an upper gap (208) and a lower gap (204). Arcuate electrodes (202, 206) and cuff (209) cooperate to define a generally circular shape, though in other versions arcuate electrodes (202, 206) and cuff (209) may cooperate to define a shape that is elliptical, oval-shaped, square, triangular, or otherwise non-circular. In the present example, the generally circular shape defined by arcuate electrodes (202, 206) and cuff (209) extends along a plane that is perpendicular to the longitudinal axis of shaft assembly (130). In some other versions, the generally circular shape (or other non-circular shape) defined by arcuate electrodes (202, 206) and cuff (209) extends along a plane that is obliquely oriented or otherwise transverse to the longitudinal axis of shaft assembly (130).

In some versions, arcuate electrodes (202, 206) may each include any one or more of a conductive wire, plate, film, and/or coating, and may be formed of any suitable material or combination of materials including but not limited to metallic conductive materials such as copper, gold, steel, aluminum, silver, nitinol, etc. and/or non-metallic conductive materials such as conducting polymers, silicides, graphite, etc. Arcuate electrodes (202, 206) may be secured and cuff (209) any suitable fashion, including but not limited to being secured via an adhesive, via vapor deposition, or otherwise. Similarly, cuff (209) may be secured to flexible portion (134) in any suitable fashion, including but not limited to being secured via an adhesive, via press-fit, via threaded coupling, or otherwise. While two arcuate electrodes (202, 206) are shown, any other suitable number of arcuate electrodes (202, 206) may be provided.

Each arcuate electrode (202, 206) is coupled with a corresponding one or more wire(s), trace(s), and/or other conductive element(s) that electrically couple arcuate electrodes (202, 206) with electrical generator (102). In versions where flexible portion (134) of shaft assembly (130) is formed of an electrically conductive material, cuff (209) may be formed of an electrically insulative material such that cuff (209) electrically isolates arcuate electrodes (202, 206) relative to flexible portion (134). Insulative properties of cuff (209) may also prevent the formation of short circuits between arcuate electrodes (202, 206). With electrodes (202, 206) being coupled with electrical generator (102), electrodes (202, 206) are operable to apply electrical energy to tissue contacting arcuate electrodes (202, 206). In some versions, arcuate electrodes (202, 206) are provided at different polarities, such that arcuate electrodes (202, 206) are operable to apply bipolar electrical energy to tissue contacting arcuate electrodes (202, 206). In some other versions (e.g., where the patient is in contact with a ground pad), arcuate electrodes (202, 206) are operable to apply monopolar electrical energy to tissue contacting arcuate electrodes (202, 206).

In some scenarios, the electrical energy (or other electrical energy) from arcuate electrodes (202, 206) is used to provide ablation, provide electroporation, or otherwise treat tissue. In some other scenarios, the electrical energy (or other electrical energy) is used to provide nerve stimulation or other effects. In addition to applying electrical energy (or other electrical energy) to tissue, or as an alternative to applying electrical energy (or other electrical energy) to tissue, arcuate electrodes (202, 206) may be used to pick up potentials from tissue, sense impedance of tissue, and/or provide other sensing capabilities.

Needle electrodes (222, 226) are positioned at distal ends of respective shafts (220, 224), such that needle electrode (222) is at the distal end of shaft (220) and needle electrode (226) is at the distal end of shaft (224). In the present example, shafts (220, 224) and needle electrodes (222, 226) are straight and configured to extend along or parallel to the longitudinal axis of shaft assembly (130) when needle electrodes (222, 226) are distally positioned as shown in FIG. 3B. In some versions, shafts (220, 224) and/or needle electrodes (222, 226) may be resiliently biased to splay outwardly relative to the longitudinal axis of shaft assembly (130) when needle electrodes (222, 226) are distally positioned. By way of example only, such biasing and/or outward splaying of needle electrodes (222, 226) may be provided in accordance with at least some of the teachings of U.S. Pat. App. No. 63/067,495, entitled “ENT Ablation Instrument with Electrode Loop,” filed Aug. 19, 2020.

In some versions (e.g., where shafts (220, 224) are formed of an electrically conductive material), an insulating layer or other electrically insulating barrier is interposed between each electrode (222, 226) and its respective shaft (220, 224). Shafts (220, 224) of the present example are coupled with slider (122) such that slider (122) is operable to drive needle electrodes (222, 226), via shafts (220, 224), between a proximally retracted position (FIG. 3A) and a distally extended position (FIG. 3B) as noted above. Each needle electrode (222, 226) has a blunt distal tip in the present example. In some other versions, one or both of needle electrodes (222, 226) has a sharp distal tip. Some versions of needle electrodes (222, 226) may penetrate tissue. In such versions, when second slider (122) is advanced distally, needle electrodes (222, 226) are driven to extend distally past the transverse plane defined by arcuate electrodes (202, 206), as shown in FIG. 3B. The operator may arrest distal advancement of second slider (122) at any suitable position along the length of body (112) of handle assembly (110) to achieve any suitable depth of penetration of needle electrodes (222, 226) into tissue.

Some versions of needle electrodes (222, 226) and shafts (220, 224) may also be hollow, such that needle electrodes (222, 226) and shafts (220, 224) may be used to deliver fluid (e.g., irrigation fluid, therapeutic agent, etc.). While two needle electrodes (222, 226) and shafts (220, 224) are shown, any other suitable number of needle electrodes and shafts may be provided.

Each needle electrode (222, 226) is coupled with a corresponding one or more wire(s), trace(s), and/or other conductive element(s) that electrically couple needle electrodes (222, 226) with electrical generator (102). As noted above, an insulating material may prevent needle electrodes (222, 226) from electrically energizing shafts (220, 224), which may in turn prevent shafts (220, 224) from electrically energizing flexible portion (134) of shaft assembly (130). As shown in FIG. 3B, shafts (220, 224) are configured to maintain spatial separation between needle electrodes (220, 226) thereby preventing the formation of short circuits between needle electrodes (222, 226). With needle electrodes (222, 226) being coupled with electrical generator (102), needle electrodes (222, 226) are operable to apply electrical energy to tissue contacting needle electrodes (222, 226). In some versions, needle electrodes (222, 226) are provided at different polarities, such that needle electrodes (222, 226) are operable to apply bipolar electrical energy to tissue contacting needle electrodes (222, 226). In some other versions (e.g., where the patient is in contact with a ground pad), needle electrodes (222, 226) are operable to apply monopolar electrical energy to tissue contacting needle electrodes (222, 226).

In some scenarios, the electrical energy (or other electrical energy) from needle electrodes (222, 226) is used to provide ablation, provide electroporation, or otherwise treat tissue. In some other scenarios, the electrical energy (or other electrical energy) is used to provide nerve stimulation or other effects. In addition to applying electrical energy (or other electrical energy) to tissue, or as an alternative to applying electrical energy (or other electrical energy) to tissue, needle electrodes (222, 226) may be used to pick up potentials from tissue, sense impedance of tissue, and/or provide other sensing capabilities.

In some versions, arcuate electrodes (202, 206) are configured to cooperate with each other to apply bipolar electrical energy to tissue; and needle electrodes (222, 226) are also configured to cooperate with each other to apply bipolar electrical energy to tissue. One or both of arcuate electrodes (202, 206) may also cooperate with one or both of needle electrodes (222, 226) to apply bipolar electrical energy to tissue. For instance, needle electrodes (222, 226) may together serve as an active electrode (or return electrode) while arcuate electrodes (202, 206) together serve as a return electrode (or active electrode) to provide bipolar electrical energy to tissue. As another variation, electrodes (202, 222) on one lateral half of end effector (200) may together serve as an active electrode (or return electrode) while electrodes (206, 224) on the other lateral half of end effector (200) may together serve as a return electrode (or active electrode). As yet another variation, electrodes (202, 224) may together serve as an active electrode (or return electrode) while electrodes (206, 222) may together serve as a return electrode (or active electrode). Any other suitable combinations and arrangements of polarities may be used.

When needle electrodes (222, 226) are used to deliver electrical energy to tissue, needle electrodes (222, 226) may be advanced into the tissue such that needle electrodes (222, 226) penetrate the tissue; then needle electrodes (222, 226) may be activated to apply the electrical energy to the penetrated tissue. When arcuate electrodes (202, 206) are used to deliver electrical energy to tissue, arcuate electrodes (202, 206) may be pressed against the tissue such that arcuate electrodes (202, 206) engage the tissue; then arcuate electrodes (202, 206) may be activated to apply the electrical energy to the engaged tissue.

Instrument (100) thus allows an operator to choose between applying electrical energy to a surface of tissue (e.g., via arcuate electrodes (202, 206)) and/or within penetrated tissue (e.g., via needle electrodes (222, 226)). Instrument (100) may therefore be used to perform a relatively shallow ablation (e.g., via arcuate electrodes (202, 206)), a relatively deep ablation (e.g., via needle electrodes (222, 226)), or a volumetric ablation (e.g., via arcuate electrodes (202, 206) in combination with needle electrodes (222, 226)). By way of further example only, instrument (100) may be used to perform a vidian neurectomy, a posterior nasal neurectomy, a turbinate reduction, or any other suitable procedure. In some cases, a combination of arcuate electrodes (202, 206) and needle electrodes (222, 226) may be used to perform a turbinate reduction. Other suitable ways in which needle electrodes (222, 226) and/or arcuate electrodes (202, 206) may be used to apply electrical energy to tissue will be apparent to those skilled in the art in view of the teachings herein.

### C. Example of Camera Assembly

As noted above, instrument (100) of the present example further includes a camera assembly (210), which forms art of end effector (200) at distal end (136) of shaft assembly (130). Camera assembly (210) is operable to provide visualization at a target tissue site distal to distal end (136). Camera assembly (210) of this example includes a camera (212) and a pair of illuminating elements (214) laterally flanking camera (212). Camera (212) may be in the form of a camera that is suitably sized to fit within shaft assembly (130) while still permitting space for needle electrodes (222, 226) and shafts (220, 224) in shaft assembly (130).

Illuminating elements (214) are configured and operable to illuminate the field of view of camera (212). While two illuminating elements (214) are used in the present example, other versions may employ just one illuminating element (214) or more than two illuminating elements (214). In the present example, illuminating elements (214) include LEDs. In some other versions, illuminating elements (214) include fiber optic components. For instance, each illuminating element (214) may include a lens that is optically coupled with one or more respective optical fibers or optical fiber bundles. Such optical fibers or optical fiber bundles may extend along shaft assembly (130) and be optically coupled with a source of light that is either integrated into handle assembly (110) (or some other body from which shaft assembly (130) extends) or otherwise provided.

Regardless of the form taken by illuminating elements (214), in some versions illuminating elements (214) are driven to emit light at one or more wavelengths selected to facilitate visualization of a tissue state. For instance, one or both of illuminating elements (214) may be driven to emit light at a wavelength associated with the color of tissue that has been sufficiently ablated. In some such versions, the light may provide visual emphasis to the operator to assist the operator in visually confirming that the ablation is complete. In addition, or in the alternative, one or both of illuminating elements (214) may be driven to emit light at a wavelength associated with the color of tissue that should be ablated. As another example, some versions may provide selectable variation of the wavelength of light emitted by one or both of illuminating elements (214), such that the wavelength may be varied based on operator selection and/or based on the stage of the procedure. For instance, one or more sensors (e.g., tissue impedance detectors, thermistors, etc.) may provide real-time feedback on the state of the target tissue; and this feedback may be used to automatically vary the wavelength of light emitted by one or both of illuminating elements (214). Alternatively, the light emitted by one or both of illuminating elements (214) may have any other suitable properties.

In some versions, camera assembly (210) further includes one or more fluid conduits. Such fluid conduits may be used to apply irrigation fluid (e.g., saline, etc.) to the target tissue site, to flush debris from camera (212), and/or for any other suitable purpose(s). In some scenarios, fluid expelled via camera assembly (210) may assist in promoting electrical continuity between one or more of electrodes (202, 206, 222, 226) and adjacent tissue. In addition, or in the alternative, one or more conduits of camera assembly (210) may be used to apply suction. Such suction may be applied to aspirate smoke, vapor, and/or other aspiratable results from a tissue ablation process. Such aspiration may further promote visualization during and after the ablation process by helping to clear the visual field of view for camera (212). Such suction may also draw away excess irrigation fluid. In addition to, or as an alternative to, applying suction via conduits of camera assembly (210), suction may be applied via the interior of shaft assembly (130). Alternatively, fluid communication and/or suction may be provided in any other suitable fashion. In some versions, fluid communication and/or suction are/is omitted.

As noted above, camera assembly (210) may be driven longitudinally relative to shaft assembly (130) by driving slider (120) longitudinally along handle assembly (110). The relative longitudinal movement between distal end (136) of shaft assembly (130) and camera assembly (210) may enable the operator to more readily visualize a tissue region that is targeted for ablation before the ablation occurs, visualize the targeted tissue region during ablation, and/or visualize the targeted tissue region after ablation. In some versions, slider (120) may allow the operator to drive camera assembly (210) distally past distal end (136) of shaft assembly (130). In addition, or in the alternative, slider (120) may allow the operator to retract camara assembly (210) proximally to a position that is proximal to distal end (136) of shaft assembly (130).

In versions permitting relative longitudinal movement between distal end (136) of shaft assembly (130) and camara assembly (210), an operator may wish to have distal end (136) of shaft assembly (130) and camara assembly (210) at substantially the same longitudinal position, as shown in FIGS. 3A-3B, while the operator maneuvers distal end (136) toward the targeted tissue region. Once the operator reaches the targeted tissue region and presses arcuate electrodes (202, 206) against the targeted tissue, the operator may wish to have camara assembly (210) retracted proximally relative to distal end (136) while applying the electrical energy to the tissue via arcuate electrodes (202, 206). Once the operator believes that the ablation is complete, the operator may wish to have camara assembly (210) advanced distally relative to distal end (136), to better visualize the ablated tissue to confirm that they are satisfied with the ablation. Other suitable ways in which an operator may wish to utilize instrument (100) with camara assembly (210) at different longitudinal positions relative to distal end (136) of shaft assembly (130) will be apparent to those skilled in the art in view of the teachings herein.

In some other versions, the longitudinal position of camera (210) is fixed relative to shaft (130), such that slider (120) is omitted or used for some other purpose. By way of further example only, camera assembly (210) may be configured and operable in accordance with at least some of the teachings of U.S. Pub. No. 2022/0054188, entitled “ENT Ablation Instrument with Electrode Loop,” published Feb. 24, 2022, the disclosure of which is incorporated by reference herein, in its entirety.

### D. Example of Alternative End Effector

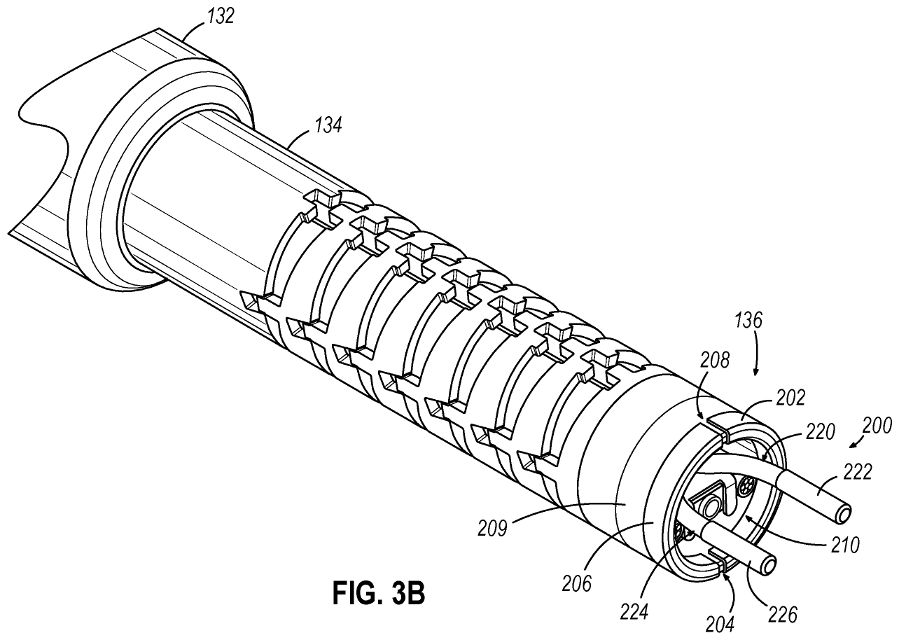

FIG. 4 shows an example of another end effector (300) that may be incorporated into distal end (136) of shaft assembly (130), in place of end effector (200) described above. Shaft assembly (130) and the rest of instrument (100) may still be configured and operable just like the example described above, such that overlapping details will not be reiterated below. End effector (300) of this example includes six electrodes (310, 314, 318, 322, 326, 330). Each electrode (310, 314, 318, 322, 326, 330) of this example is in the form of a distally-facing, arcuate conductive element (e.g., metal) that is fixedly secured relative to distal end (136) of shaft assembly (130) via a cuff (309). Electrodes (310, 318, 322, 330) have the same length as each other, such that electrodes (310, 318, 322, 330) extend along the same angular extent. Electrodes (314, 326) have the same length as each other, such that electrodes (314, 326) extend along the same angular extent. Electrodes (310, 318, 322, 330) are longer than electrodes (314, 326) in this example.

Electrode (310) is angularly offset from electrode (322) by 180 degrees. Electrode (314) is angularly offset from electrode (326) by 180 degrees. Electrode (318) is angularly offset from electrode (330) by 180 degrees. Electrodes (310, 314) are angularly separated from each other by a gap (312). Electrodes (314, 318) are angularly separated from each other by a gap (316). Electrodes (318, 322) are angularly separated from each other by a gap (320). Electrodes (322, 326) are angularly separated from each other by a gap (324). Electrodes (326, 330) are angularly separated from each other by a gap (320). Electrodes (330, 310) are angularly separated from each other by a gap (332).

Electrodes (310, 314, 318, 322, 326, 330) and cuff (309) cooperate to define a generally circular shape, though in other versions electrodes (310, 314, 318, 322, 326, 330) and cuff (309) may cooperate to define a shape that is elliptical, oval-shaped, square, triangular, or otherwise non-circular. In the present example, the generally circular shape defined by electrodes (310, 314, 318, 322, 326, 330) and cuff (309) extends along a plane that is perpendicular to the longitudinal axis of shaft assembly (130). In some other versions, the generally circular shape (or other non-circular shape) defined by electrodes (310, 314, 318, 322, 326, 330) and cuff (309) extends along a plane that is obliquely oriented or otherwise transverse to the longitudinal axis of shaft assembly (130).

In some versions, electrodes (310, 314, 318, 322, 326, 330) may each include any one or more of a conductive wire, plate, film, and/or coating, and may be formed of any suitable material or combination of materials including but not limited to metallic conductive materials such as copper, gold, steel, aluminum, silver, nitinol, etc. and/or non-metallic conductive materials such as conducting polymers, silicides, graphite, etc. Electrodes (310, 314, 318, 322, 326, 330) may be secured and cuff (309) any suitable fashion, including but not limited to being secured via an adhesive, via vapor deposition, or otherwise. Similarly, cuff (309) may be secured to flexible portion (134) in any suitable fashion, including but not limited to being secured via an adhesive, via press-fit, via threaded coupling, or otherwise. While six electrodes (310, 314, 318, 322, 326, 330) are shown, any other suitable number of electrodes (310, 314, 318, 322, 326, 330) may be provided.

Each electrode (310, 314, 318, 322, 326, 330) is coupled with a corresponding one or more wire(s), trace(s), and/or other conductive element(s) that electrically couple electrodes (310, 314, 318, 322, 326, 330) with electrical generator (102). In versions where flexible portion (134) of shaft assembly (130) is formed of an electrically conductive material, cuff (309) may be formed of an electrically insulative material such that cuff (309) electrically isolates arcuate electrodes (310, 314, 318, 322, 326, 330) relative to flexible portion (134). Insulative properties of cuff (309) may also prevent the formation of short circuits between electrodes (310, 314, 318, 322, 326, 330). With electrodes (310, 314, 318, 322, 326, 330) being coupled with electrical generator (102), electrodes (310, 314, 318, 322, 326, 330) are operable to apply electrical energy to tissue contacting electrodes (310, 314, 318, 322, 326, 330). In some versions, electrodes (310, 314, 318, 322, 326, 330) are provided at different polarities, such that electrodes (310, 314, 318, 322, 326, 330) are operable to apply bipolar electrical energy to tissue contacting electrodes (310, 314, 318, 322, 326, 330). In some other versions (e.g., where the patient is in contact with a ground pad), electrodes (310, 314, 318, 322, 326, 330) are operable to apply monopolar electrical energy to tissue contacting electrodes (310, 314, 318, 322, 326, 330).

In some scenarios, the electrical energy (or other electrical energy) from electrodes (310, 314, 318, 322, 326, 330) is used to provide ablation, provide electroporation, or otherwise treat tissue. In some other scenarios, the electrical energy (or other electrical energy) is used to provide nerve stimulation or other effects. In addition to applying electrical energy (or other electrical energy) to tissue, or as an alternative to applying electrical energy (or other electrical energy) to tissue, electrodes (310, 314, 318, 322, 326, 330) may be used to pick up potentials from tissue, sense impedance of tissue, and/or provide other sensing capabilities.

### E. Examples of Other Features for Instrument

As noted above, various electrical circuit components may be integrated into shaft assembly (130), regardless of whether end effector (200) or end effector (300) is positioned at distal end (136) of shaft assembly (130). Such electrical circuit components may include flex circuits and various other kinds of components. By way of example only, shaft assembly (130) and end effector (200, 300) may include any of the various features described in U.S. patent application Ser. No. 17/584,693, entitled “Flexible Sensor Assembly for ENT Instrument,” filed Jan. 26, 2022, the disclosure of which is incorporated by reference herein, in its entirety.

While not shown, instrument (100) may also include one or more position sensors that are operable to generate signals indicative of the position of end effector (200) and/or or some other component(s) of instrument (100) in three-dimensional space. Such a position sensor may be integrated directly into shaft assembly (130) or elsewhere into instrument (100). In addition, or in the alternative, such a position sensor may be integrated into a guidewire or other component that is disposed in shaft assembly (130). Such a position sensor may take the form of one or more coils that generate signals in response to the presence of an alternating magnetic field. The position data generated by such position signals may be processed by a system that provides a visual indication to the operator to show the operator where end effector and/or some other component(s) of instrument (100) is located within the patient in real time. Such a visual indication may be provided as an overlay on one or more preoperatively obtained images (e.g., CT scans) of the patient's anatomy. Such position sensing and navigation capabilities may be provided in accordance with at least some of the teachings of the various references cited herein.

### III. Example of Nerve Stimulation to Test Denervation Success

In procedures where an instrument like instrument (100) is used to perform a denervation procedure, it may be desirable to perform a test shortly after attempting the denervation, to confirm whether the denervation was successful. It may be further desirable to use the same instrument that was used for the denervation attempt to test the success of the denervation attempt. This may avoid the need for using one instrument to perform the denervation procedure and another, separate instrument for testing the success of the denervation procedure. As described below, an instrument like instrument (100) may be used to first perform a denervation procedure and then test the success of the denervation procedure, without needing to withdraw instrument (100) from the patient between the denervation attempt and the denervation test. Such denervation performance and testing may be performed regardless of whether instrument is equipped with end effector (200) or end effector (300).

In versions where instrument (100) is equipped with end effector (200), an operator may navigate shaft assembly (130) into the nasal cavity (10) of a patient and position end effector (200) adjacent to a target tissue site (e.g., posterior nasal nerve (40), vidian nerve (34), etc.). Such positioning of end effector (200) may be done with visual guidance from camera assembly (210). In addition, or in the alternative, such positioning of end effector (200) may be done with guidance from one or more position sensors positioned in end effector (200) and/or in shaft assembly (130) and/or elsewhere in instrument (100).

Once end effector (200) is positioned adjacent to a target tissue site, arcuate electrodes (202, 206) may be pressed against the tissue and activated to apply electrical energy to the tissue. In some versions, arcuate electrodes (202, 206) are activated at opposing polarities to apply bipolar electrical energy to the tissue. In some other versions, arcuate electrodes (202, 206) are activated at the same polarity and cooperate with a ground pad contacting the patient to apply monopolar electrical energy to the tissue. In either case, the electrical energy may cause ablation of the tissue. In scenarios where the target site includes a nerve (e.g., posterior nasal nerve (40), vidian nerve (34), etc.), the electrical energy may provide denervation by effectively destroying the portion of the nerve at the target site.

After the electrical energy has been applied to tissue via arcuate electrodes (202, 206), needle electrodes (222, 226) may be advanced distally from the position shown in FIG. 3A to the position shown in FIG. 3B. In some scenarios, this causes needle electrodes (222, 226) to penetrate the tissue. With needle electrodes (222, 226) disposed in the tissue or otherwise communicating with the tissue, needle electrodes (222, 226) may be used to test whether the electrical denervation by arcuate electrodes (202, 206) was successful. To complete such testing, one needle electrode (222) may apply an electrical stimulus signal to the nerve while the other needle electrode (226) serves as a sensor to detect whether a signal indicating a nerve response to the stimulus signal. In some other versions, both needle electrodes (222, 226) provide stimulus and sensing capabilities (e.g., using bidirectional multiplexing techniques). In either case, end effector (200) may be positioned such that one needle electrode (222) is positioned on one side of the targeted nerve while the other needle electrode (226) is positioned on the other side of the targeted nerve. As noted above, position sensors may assist in providing such positioning of needle electrodes (222, 226) in relation to the targeted nerve. A controller that includes electrical generator (102) may be used to generate the stimulus signal and process the response. The stimulus signal may be electrical but need not necessarily be within the RF range.

If the sensing needle electrode (226) detects successful transmission of the stimulus signal from the stimulating needle electrode (222), such successful transmission may indicate that the denervation procedure was not successful. The controller may notify the operator accordingly. In such scenarios, the operator may again apply electrical energy to the tissue via arcuate electrodes (202, 206); and needle electrodes (222, 226) may again be used to detect whether the next attempt at denervation was successful. This process may be repeated until the sensing needle electrode (226) fails to detect transmission of the stimulus signal from the stimulating needle electrode (222). Such a failure of sensing needle electrode (226) to detect transmission of the stimulus signal from the stimulating needle electrode (222) may indicate that the denervation procedure was successful. Again, the controller may notify the operator accordingly. At that point, shaft assembly (130) may be moved to reposition end effector (200) at another target site; or may be removed from the nasal cavity (10) altogether.

In versions where instrument (100) is equipped with end effector (300), an operator may navigate shaft assembly (130) into the nasal cavity (10) of a patient and position end effector (300) adjacent to a target tissue site (e.g., posterior nasal nerve (40), vidian nerve (34), etc.). Such positioning of end effector (300) may be done with visual guidance from camera assembly (210). In addition, or in the alternative, such positioning of end effector (300) may be done with guidance from one or more position sensors positioned in end effector (300) and/or in shaft assembly (130) and/or elsewhere in instrument (100).

Once end effector (230) is positioned adjacent to a target tissue site, electrodes (310, 318, 322, 330) may be pressed against the tissue and activated to apply electrical energy to the tissue. In some versions, electrodes (310, 318, 322, 330) are activated at opposing polarities to apply bipolar electrical energy to the tissue. In some other versions, electrodes (310, 318, 322, 330) are activated at the same polarity and cooperate with a ground pad contacting the patient to apply monopolar electrical energy to the tissue. In either case, the electrical energy may cause ablation of the tissue. In scenarios where the target site includes a nerve (e.g., posterior nasal nerve (40), vidian nerve (34), etc.), the electrical energy may provide denervation by effectively destroying the portion of the nerve at the target site.

After the electrical energy has been applied to tissue via electrodes (310, 318, 322, 330), electrodes (314, 326) may be used to test whether the electrical denervation by arcuate electrodes (202, 206) was successful. To complete such testing, one electrode (314) may apply an electrical stimulus signal to the nerve while the other needle electrode (326) serves as a sensor to detect whether a signal indicating a nerve response to the stimulus signal. In some other versions, both electrodes (314, 326) provide stimulus and sensing capabilities (e.g., using bidirectional multiplexing techniques). In either case, end effector (300) may be positioned such that one electrode (314) is positioned on one side of the targeted nerve while the other electrode (326) is positioned on the other side of the targeted nerve. As noted above, position sensors may assist in providing such positioning of electrodes (314, 326) in relation to the targeted nerve. A controller that includes electrical generator (102) may be used to generate the stimulus signal and process the response. The stimulus signal may be electrical but need not necessarily be within the RF range.

If the sensing electrode (326) detects successful transmission of the stimulus signal from the stimulating electrode (314), such successful transmission may indicate that the denervation procedure was not successful. The controller may notify the operator accordingly. In such scenarios, the operator may again apply electrical energy to the tissue via electrodes (310, 318, 322, 330); and electrodes (314, 326) may again be used to detect whether the next attempt at denervation was successful. This process may be repeated until the sensing electrode (326) fails to detect transmission of the stimulus signal from the stimulating electrode (314). Such a failure of sensing electrode (326) to detect transmission of the stimulus signal from the stimulating electrode (314) may indicate that the denervation procedure was successful. Again, the controller may notify the operator accordingly. At that point, shaft assembly (130) may be moved to reposition end effector (300) at another target site; or may be removed from the nasal cavity (10) altogether.

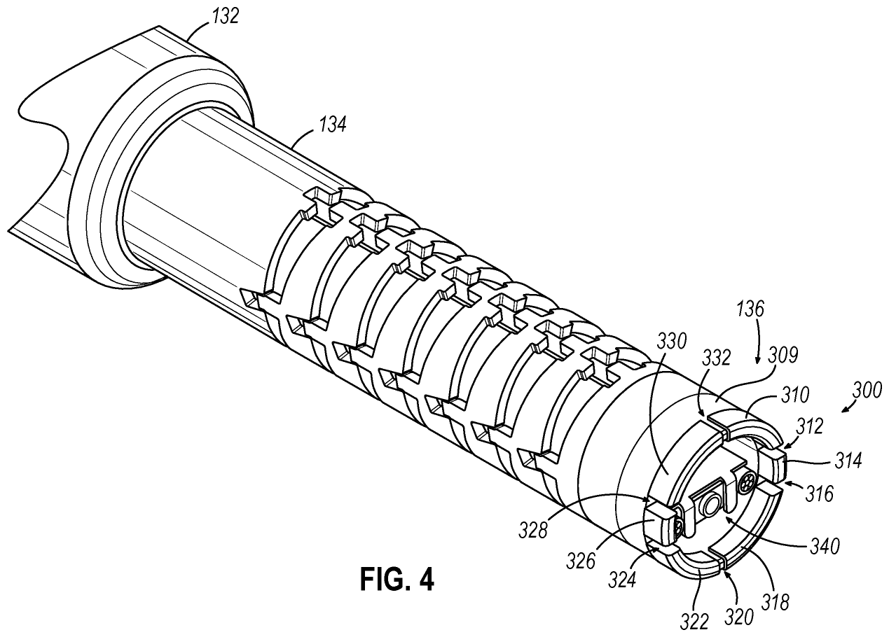

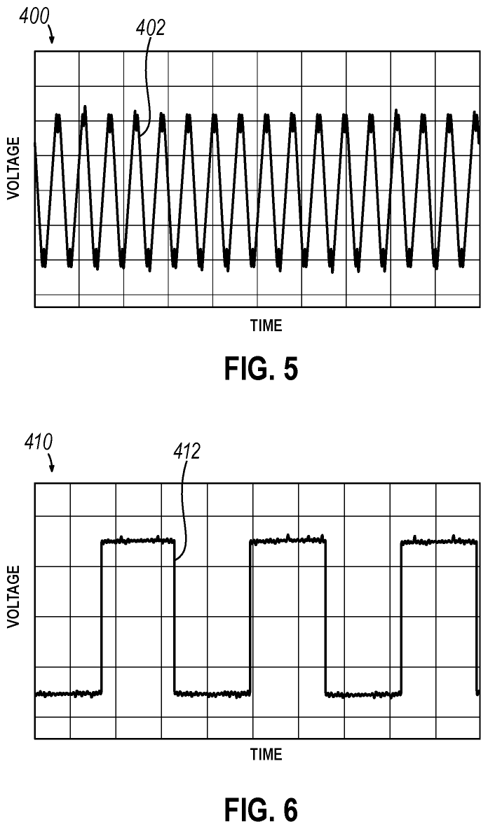

As noted above, needle electrodes (222, 226) of end effector (200) or electrodes (314, 326) of end effector (200) may be used to determine whether a targeted nerve has been successfully denervated. In such a process, a nerve stimulation signal may take various forms. FIG. 5 shows a graph (400) of a relatively high frequency signal (402). This high frequency signal (402) may be effective in penetrating tissue (e.g., mucosa, etc.) without losing amplitude prior to the signal (402) reaching the nerve underlying the tissue. To the extent that the high frequency of signal (402) promotes penetration of tissue to reach an underlying nerve, the high frequency may not necessarily be ideal for stimulating the nerve. By contrast, FIG. 6 shows a graph (410) of a relatively low frequency signal (412) having a square waveform. In some cases, such a relatively low frequency, square-waveform signal (412) may be effective in stimulating a nerve. However, such a relatively low frequency, square-waveform signal (412) may not necessarily be ideal for penetrating tissue to reach an underlying nerve.

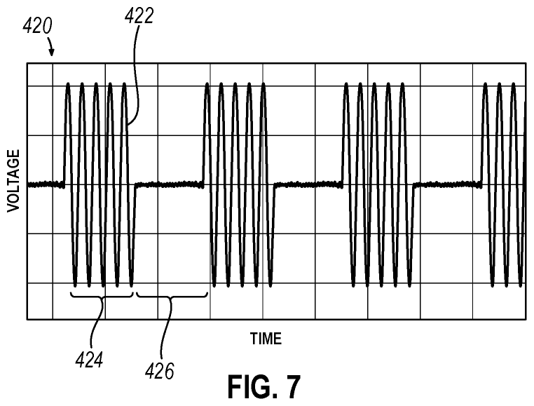

It may therefore be desirable to provide a nerve stimulus signal that provides a combination of the tissue penetrating capabilities of signal (402) and the nerve stimulating capabilities of signal (412). To that end, FIG. 7 shows a graph (420) of a signal (422) representing a version of signal (402) that is modulated with signal (412). Signal (422) thus provides pockets (424) of the high frequency of signal (402), separated by gaps (426) that effectively provide pockets (424) at the same frequency as signal (412). In other words, the high frequency signal is applied through a square wave duty cycle and amplitude that effectively pulses the high frequency signal. Such a waveform may effectively combine the tissue penetrating capabilities of signal (402) and the nerve stimulating capabilities of signal (412), which may in turn result in highly efficient and efficacious application of electrical stimulus to the targeted nerve. As noted above, signal (422) may be applied to the targeted nerve via needle electrodes (222, 226) of end effector (200), via electrodes (314, 326) of end effector (200), or via any other suitable kind(s) of electrodes. As also noted above, the targeted nerve may include a posterior nasal nerve (40), a vidian nerve (34), or any other suitable kind of nerve.

A controller that includes electrical generator (102) may further include components that may be used to generate signal (422). For instance, the controller may include a first waveform generator that generates a waveform like the waveform of signal (402), a second waveform generator that generates a waveform like the waveform of signal (412), and a modulator that generates a signal like signal (422) by modulating the waveform of the first waveform generator with the waveform of the second waveform generator.

### IV. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a shaft assembly having a distal end, the shaft assembly being configured to fit in a nasal cavity of a patient, the shaft assembly defining a longitudinal axis; (b) a first electrode assembly at the distal end of the shaft assembly; (c) a second electrode assembly at the distal end of the shaft assembly, the second electrode assembly including: (i) a stimulus electrode, and (ii) a sensing electrode, the stimulus and sensing electrodes being positioned on opposing lateral sides in relation to the longitudinal axis of the shaft assembly; and (d) a controller, the controller being operable to: (i) generate an electrical signal to perform one or both of tissue ablation or denervation of a targeted nerve via the first electrode assembly, (ii) generate an electrical stimulus signal to stimulate the targeted nerve via the stimulus electrode of the second electrode assembly, and (iii) process a response signal received from the targeted nerve via the sensing electrode of the second electrode assembly.

### Example 2

The apparatus of Example 1, the shaft assembly having a rigid portion and a flexible portion, the flexible portion including the distal end.

### Example 3

The apparatus of Example 2, further comprising an actuator, the actuator being operable to deflect the flexible portion and thereby drive the distal end laterally relative to the longitudinal axis.

### Example 4

The apparatus of any one or more of Examples 1 through 3, further comprising a camera assembly at the distal end.

### Example 5

The apparatus of any one or more of Examples 1 through 4, further comprising a position sensor, the position sensor being operable to generate a signal indicating a position of the distal end in three-dimensional space.

### Example 6

The apparatus of any one or more of Examples 1 through 5, further comprising a cuff at the distal end, the first electrode assembly being secured to the distal end via the cuff.

### Example 7

The apparatus of Example 6, the second electrode assembly being secured to the distal end via the cuff.

### Example 8

The apparatus of any one or more of Examples 6 through 7, the cuff being formed of an electrically insulative material.

### Example 9

The apparatus of any one or more of Examples 1 through 8, the first electrode assembly including a first electrode and a second electrode.

### Example 10

The apparatus of Example 9, the first electrode and the second electrode cooperating to define a generally circular shape encircling the longitudinal axis.

### Example 11

The apparatus of any one or more of Examples 9 through 10, the first electrode and the second electrode being angularly spaced apart from each other by a first gap and a second gap.

### Example 12

The apparatus of any one or more of Examples 9 through 11, the first electrode and the second electrode each having an arcuate shape.

### Example 13

The apparatus of any one or more of Examples 9 through 12, the first electrode and the second electrode facing distally away from the distal end of the shaft assembly.

### Example 14

The apparatus of Example 13, the distal end of the shaft assembly defining a distally facing circumferential edge, the first electrode and the second electrode being positioned at the distally facing circumferential edge of the distal end of the shaft assembly.

### Example 15

The apparatus of any one or more of Examples 9 through 14, the first electrode and the second electrode being operable to apply bipolar electrical energy to tissue.

### Example 16

The apparatus of any one or more of Examples 1 through 15, the second electrode assembly further including: (i) a first shaft, the stimulus electrode being secured to the first shaft, and (ii) a second shaft, the sensing electrode being secured to the second shaft.

### Example 17

The apparatus of Example 16, the first shaft being parallel with the second shaft.

### Example 18

The apparatus of Example 17, the first shaft and the second shaft both being parallel with the longitudinal axis.

### Example 19

The apparatus of any one or more of Examples 1 through 18, further comprising an actuator, the actuator being operable to drive the second electrode assembly longitudinally relative to the shaft assembly.

### Example 20

The apparatus of Example 19, the actuator being operable to drive the second electrode assembly from a proximal position to a distal position, the stimulus electrode and the sensing electrode being positioned proximally in relation to the distal end of the shaft assembly when the second electrode assembly is in the proximal position, the stimulus electrode and the sensing electrode being positioned distally in relation to the distal end of the shaft assembly when the second electrode assembly is in the distal position.

### Example 21

The apparatus of any one or more of Examples 1 through 20, the stimulus electrode comprising a first needle electrode, the sensing electrode comprising a second needle electrode.

### Example 22

The apparatus of any one or more of Examples 1 through 21, the first electrode assembly including a first electrode and a second electrode, the stimulus electrode being angularly interposed between the first electrode and the second electrode.

### Example 23

The apparatus of Example 22, the sensing electrode being angularly interposed between the second electrode and the first electrode.

### Example 24

The apparatus of Example 23, the first electrode assembly further including a third electrode and a fourth electrode, the third electrode being angularly interposed between the second electrode and the sensing electrode, the fourth electrode being angularly interposed between the sensing electrode and the first electrode.

### Example 25

The apparatus of Example 24, the first electrode, the second electrode, the third electrode, the fourth electrode, the stimulus electrode, and the sensing electrode cooperating to define a generally circular shape encircling the longitudinal axis.

### Example 26

The apparatus of any one or more of Examples 22 through 25, the stimulus electrode and the sensing electrode facing distally away from the distal end of the shaft assembly.

### Example 27

The apparatus of Example 26, the distal end of the shaft assembly defining a distally facing circumferential edge, the stimulus electrode and the sensing electrode being positioned at the distally facing circumferential edge of the distal end of the shaft assembly.

### Example 28

The apparatus of any one or more of Examples 1 through 27, the controller being further operable to generate an electrical stimulus signal having a modulated waveform.

### Example 29

The apparatus of Example 28, the modulated waveform providing pulsed high frequency signals.

### Example 30

The apparatus of Example 29, the modulated waveform providing high frequency signal pockets applied through a square wave duty cycle.

### Example 31

An apparatus, comprising: (a) a shaft assembly having a distal end, the shaft assembly being configured to fit in a nasal cavity of a patient, the shaft assembly defining a longitudinal axis; (b) a first electrode assembly at the distal end of the shaft assembly, the first electrode assembly including a distally-facing first electrode; (c) a second electrode assembly at the distal end of the shaft assembly, the second electrode assembly including: (i) a stimulus electrode, and (ii) a sensing electrode; and (d) a controller, the controller being operable to: (i) generate an electrical signal to perform one or both of tissue ablation or denervation of a targeted nerve via the first electrode assembly, (ii) generate an electrical stimulus signal to stimulate the targeted nerve via the stimulus electrode of the second electrode assembly, and (iii) process a response signal received from the targeted nerve via the sensing electrode of the second electrode assembly.

### Example 32

The apparatus of Example 31, the shaft assembly having a rigid portion and a flexible portion, the flexible portion including the distal end.

### Example 33

The apparatus of Example 32, further comprising an actuator, the actuator being operable to deflect the flexible portion and thereby drive the distal end laterally relative to the longitudinal axis.

### Example 34

The apparatus of any one or more of Examples 31 through 33, further comprising a camera assembly at the distal end.

### Example 35

The apparatus of any one or more of Examples 31 through 34, further comprising a position sensor, the position sensor being operable to generate a signal indicating a position of the distal end in three-dimensional space.

### Example 36

The apparatus of any one or more of Examples 31 through 35, further comprising a cuff at the distal end, the first electrode assembly being secured to the distal end via the cuff.

### Example 37

The apparatus of Example 36, the second electrode assembly being secured to the distal end via the cuff.

### Example 38

The apparatus of any one or more of Examples 36 through 37, the cuff being formed of an electrically insulative material.

### Example 39

The apparatus of any one or more of Examples 31 through 38, the first electrode assembly including a second electrode.

### Example 40

The apparatus of Example 39, the first electrode and the second electrode cooperating to define a generally circular shape encircling the longitudinal axis.

### Example 41

The apparatus of any one or more of Examples 39 through 40, the first electrode and the second electrode being angularly spaced apart from each other by a first gap and a second gap.

### Example 42

The apparatus of any one or more of Examples 39 through 41, the first electrode and the second electrode each having an arcuate shape.

### Example 43

The apparatus of any one or more of Examples 39 through 42, the distal end of the shaft assembly defining a distally facing circumferential edge, the first electrode and the second electrode being positioned at the distally facing circumferential edge of the distal end of the shaft assembly.

### Example 44

The apparatus of any one or more of Examples 39 through 43, the first electrode and the second electrode being operable to apply bipolar electrical energy to tissue.

### Example 45

The apparatus of any one or more of Examples 31 through 44, the second electrode assembly further including: (i) a first shaft, the stimulus electrode being secured to the first shaft, and (ii) a second shaft, the sensing electrode being secured to the second shaft.

### Example 46

The apparatus of Example 45, the first shaft being parallel with the second shaft.

### Example 47

The apparatus of Example 46, the first shaft and the second shaft both being parallel with the longitudinal axis.

### Example 48

The apparatus of any one or more of Examples 31 through 47, further comprising an actuator, the actuator being operable to drive the second electrode assembly longitudinally relative to the shaft assembly.

### Example 49

The apparatus of Example 48, the actuator being operable to drive the second electrode assembly from a proximal position to a distal position, the stimulus electrode and the sensing electrode being positioned proximally in relation to the distal end of the shaft assembly when the second electrode assembly is in the proximal position, the stimulus electrode and the sensing electrode being positioned distally in relation to the distal end of the shaft assembly when the second electrode assembly is in the distal position.

### Example 50

The apparatus of any one or more of Examples 31 through 49, the stimulus electrode comprising a first needle electrode, the sensing electrode comprising a second needle electrode.

### Example 51

The apparatus of any one or more of Examples 31 through 50, the first electrode assembly including a first electrode and a second electrode, the stimulus electrode being angularly interposed between the first electrode and the second electrode.

### Example 52

The apparatus of Example 51, the sensing electrode being angularly interposed between the second electrode and the first electrode.

### Example 53

The apparatus of Example 52, the first electrode assembly further including a third electrode and a fourth electrode, the third electrode being angularly interposed between the second electrode and the sensing electrode, the fourth electrode being angularly interposed between the sensing electrode and the first electrode.

### Example 54

The apparatus of Example 53, the first electrode, the second electrode, the third electrode, the fourth electrode, the stimulus electrode, and the sensing electrode cooperating to define a generally circular shape encircling the longitudinal axis.

### Example 55

The apparatus of any one or more of Examples 51 through 54, the stimulus electrode and the sensing electrode facing distally away from the distal end of the shaft assembly.

### Example 56

The apparatus of Example 55, the distal end of the shaft assembly defining a distally facing circumferential edge, the stimulus electrode and the sensing electrode being positioned at the distally facing circumferential edge of the distal end of the shaft assembly.

### Example 57

The apparatus of any one or more of Examples 31 through 56, the controller being further operable to generate an electrical stimulus signal having a modulated waveform.

### Example 58

The apparatus of Example 57, the modulated waveform providing pulsed high frequency signals.

### Example 59

The apparatus of Example 58, the modulated waveform providing high frequency signal pockets applied through a square wave duty cycle.

### Example 60

The apparatus of any one or more of Examples 31 through 59, the stimulus and sensing electrodes being positioned on opposing lateral sides in relation to the longitudinal axis of the shaft assembly.

### Example 61

A method comprising: (a) inserting a shaft assembly into a nasal cavity of a patient, the shaft assembly defining a longitudinal axis; (b) engaging tissue of the patient with a first electrode assembly; (c) applying electrical energy to the tissue via the first electrode assembly; (d) engaging the tissue with a second electrode assembly; (e) applying an electrical stimulus to a nerve associated with the tissue, the electrical stimulus being applied via a stimulus electrode of the second electrode assembly, the stimulus electrode contacting the tissue at a first lateral side of the longitudinal axis; and (f) determining whether a signal is received via a sensing electrode of the second electrode assembly in response to the electrical stimulus applied via the stimulus electrode, the sensing electrode contacting the tissue at a second lateral side of the longitudinal axis.

### Example 62

The method of Example 61, further comprising deflecting a distal portion of the shaft assembly laterally relative to the longitudinal axis.

### Example 63

The method of Example 62, the deflecting being performed before the inserting.

### Example 64

The method of any one or more of Examples 62 through 63, further comprising visualizing at least a portion of the nasal cavity via a camera assembly in the shaft assembly.

### Example 65

The method of any one or more of Examples 61 through 64, further comprising tracking movement of a distal end of the shaft assembly through the nasal cavity, the movement being tracked based on signals generated by one or more position sensors.

### Example 66

The method of any one or more of Examples 61 through 65, the first electrode assembly including at least one distally facing electrode secured to a distal end of the shaft assembly.

### Example 67

The method of any one or more of Examples 61 through 66, the first electrode assembly including at least two electrodes.

### Example 68

The method of Example 67, the applying electrical energy to the tissue via the first electrode assembly including applying bipolar electrical energy to the tissue.

### Example 69

The method of any one or more of Examples 61 through 68, the second electrode assembly further including: (i) a first shaft, the stimulus electrode being secured to the first shaft, and (ii) a second shaft, the sensing electrode being secured to the second shaft.

### Example 70

The method of Example 69, engaging the tissue with a second electrode assembly including driving the first and second shafts distally relative to the shaft assembly.

### Example 71

The method of Example 70, the stimulus electrode and the sensing electrode being positioned proximally in relation to a distal end of the shaft assembly before driving the first and second shafts distally relative to the shaft assembly, the stimulus electrode and the sensing electrode being positioned distally in relation to a distal end of the shaft assembly after driving the first and second shafts distally relative to the shaft assembly.

### Example 72

The method of any one or more of Examples 61 through 71, engaging the tissue with a second electrode assembly including driving the stimulus electrode and the sensing electrode into the tissue such that the stimulus electrode and the sensing electrode penetrate the tissue.

### Example 73

The method of any one or more of Examples 61 through 72, applying an electrical stimulus including applying an electrical stimulus signal having a modulated waveform.

### Example 74

The method of Example 73, the modulated waveform providing pulsed high frequency signals.

### Example 75

The method of Example 74, the modulated waveform providing high frequency signal pockets applied through a square wave duty cycle.

### Example 76

The method of any one or more of Examples 61 through 75, the nerve including a posterior nasal nerve.

### Example 77

The method of any one or more of Examples 61 through 75, the nerve including a vidian nerve.

### Example 78

The method of any one or more of Examples 61 through 77, determining whether a signal is received via the sensing electrode including determining that a signal is received via the sensing electrode, the received signal indicating that the nerve has not been denervated, the method further comprising repeating the acts of applying electrical energy to the tissue, applying an electrical stimulus to a nerve associated with the tissue, and determining whether the signal is received via a sensing electrode.

### Example 79

The method of any one or more of Examples 61 through 78, applying electrical energy to the tissue resulting in denervation of the nerve.

### Example 80

The method of Example 79, determining whether a signal is received via the sensing electrode including determining that a signal is not received via the sensing electrode, the non-receipt of the signal indicating that the nerve was denervated.

### Example 81

A method comprising: (a) inserting a shaft assembly into a nasal cavity of a patient, the shaft assembly defining a longitudinal axis; (b) engaging tissue of the patient with a first electrode assembly, the first electrode assembly including at least one distally facing electrode secured to a distal end of the shaft assembly; (c) applying electrical energy to the tissue via the at least one distally facing electrode of the first electrode assembly; (d) engaging the tissue with a second electrode assembly; (e) applying an electrical stimulus to a nerve associated with the tissue, the electrical stimulus being applied via a stimulus electrode of the second electrode assembly; and (f) determining whether a signal is received via a sensing electrode of the second electrode assembly in response to the electrical stimulus applied via the stimulus electrode.

### Example 82

The method of Example 81, further comprising deflecting a distal portion of the shaft assembly laterally relative to the longitudinal axis.

### Example 83

The method of Example 82, the deflecting being performed before the inserting.

### Example 84

The method of any one or more of Examples 82 through 83, further comprising visualizing at least a portion of the nasal cavity via a camera assembly in the shaft assembly.

### Example 85

The method of any one or more of Examples 81 through 84, further comprising tracking movement of a distal end of the shaft assembly through the nasal cavity, the movement being tracked based on signals generated by one or more position sensors.

### Example 86

The method of any one or more of Examples 81 through 85, the first electrode assembly including at least two electrodes.

### Example 87

The method of Example 86, the applying electrical energy to the tissue via the first electrode assembly including applying bipolar electrical energy to the tissue.

### Example 88

The method of any one or more of Examples 81 through 87, the second electrode assembly further including: (i) a first shaft, the stimulus electrode being secured to the first shaft, and (ii) a second shaft, the sensing electrode being secured to the second shaft.

### Example 89

The method of Example 88, engaging the tissue with a second electrode assembly including driving the first and second shafts distally relative to the shaft assembly.

### Example 90

The method of Example 89, the stimulus electrode and the sensing electrode being positioned proximally in relation to a distal end of the shaft assembly before driving the first and second shafts distally relative to the shaft assembly, the stimulus electrode and the sensing electrode being positioned distally in relation to a distal end of the shaft assembly after driving the first and second shafts distally relative to the shaft assembly.

### Example 91

The method of any one or more of Examples 81 through 90, engaging the tissue with a second electrode assembly including driving the stimulus electrode and the sensing electrode into the tissue such that the stimulus electrode and the sensing electrode penetrate the tissue.

### Example 92

The method of any one or more of Examples 81 through 91, applying an electrical stimulus including applying an electrical stimulus signal having a modulated waveform.

### Example 93

The method of Example 92, the modulated waveform providing pulsed high frequency signals.

### Example 94

The method of Example 93, the modulated waveform providing high frequency signal pockets applied through a square wave duty cycle.

### Example 95

The method of any one or more of Examples 81 through 94, the nerve including a posterior nasal nerve.

### Example 96

The method of any one or more of Examples 81 through 94, the nerve including a vidian nerve.

### Example 97

The method of any one or more of Examples 81 through 96, determining whether a signal is received via the sensing electrode including determining that a signal is received via the sensing electrode, the received signal indicating that the nerve has not been denervated, the method further comprising repeating the acts of applying electrical energy to the tissue, applying an electrical stimulus to a nerve associated with the tissue, and determining whether the signal is received via a sensing electrode.

### Example 98

The method of any one or more of Examples 81 through 97, applying electrical energy to the tissue resulting in denervation of the nerve.

### Example 99

The method of Example 98, determining whether a signal is received via the sensing electrode including determining that a signal is not received via the sensing electrode, the non-receipt of the signal indicating that the nerve was denervated.

### Example 100

The method of any one or more of Examples 81 through 99, the stimulus electrode contacting the tissue at a first lateral side of the longitudinal axis, the sensing electrode contacting the tissue at a second lateral side of the longitudinal axis.

### Example 101

An apparatus, comprising: (a) a shaft assembly having a distal end, the shaft assembly being configured to fit in a nasal cavity of a patient, the shaft assembly defining a longitudinal axis; (b) a first electrode assembly at the distal end of the shaft assembly, the first electrode assembly being operable to apply electrical energy to tissue to thereby perform one or both of tissue ablation or denervation of a targeted nerve; and (c) a second electrode assembly at the distal end of the shaft assembly, the second electrode assembly including: (i) a stimulus electrode, the stimulus electrode being operable to apply an electrical stimulus signal to stimulate the targeted nerve, and (ii) a sensing electrode, the sensing electrode being operable to receive a response signal from the targeted nerve, the stimulus and sensing electrodes being positioned on opposing lateral sides in relation to the longitudinal axis of the shaft assembly.

### Example 102

The apparatus of Example 101, further comprising a controller, the controller being operable to: (i) generate an electrical signal to perform one or both of tissue ablation or denervation of a targeted nerve via the first electrode assembly, (ii) generate an electrical stimulus signal to stimulate the targeted nerve via the stimulus electrode of the second electrode assembly, and (iii) process a response signal received from the targeted nerve via the sensing electrode of the second electrode assembly.

### Example 103

An apparatus, comprising: (a) a shaft assembly having a distal end, the shaft assembly being configured to fit in a nasal cavity of a patient, the shaft assembly defining a longitudinal axis; (b) a first electrode assembly at the distal end of the shaft assembly, the first electrode assembly including a distally-facing first electrode, the distally-facing first electrode being operable to apply electrical energy to tissue to thereby perform one or both of tissue ablation or denervation of a targeted nerve; and (c) a second electrode assembly at the distal end of the shaft assembly, the second electrode assembly including: (i) a stimulus electrode, the stimulus electrode being operable to apply an electrical stimulus signal to stimulate the targeted nerve, and (ii) a sensing electrode, the sensing electrode being operable to receive a response signal from the targeted nerve

### Example 104

The apparatus of Example 103, further comprising a controller, the controller being operable to: (i) generate an electrical signal to perform one or both of tissue ablation or denervation of a targeted nerve via the first electrode assembly, (ii) generate an electrical stimulus signal to stimulate the targeted nerve via the stimulus electrode of the second electrode assembly, and (iii) process a response signal received from the targeted nerve via the sensing electrode of the second electrode assembly.

V. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) a shaft assembly having a distal end, the shaft assembly being configured to fit in a nasal cavity of a patient, the shaft assembly defining a longitudinal axis;
(b) a first electrode assembly at the distal end of the shaft assembly;
(c) a second electrode assembly at the distal end of the shaft assembly, the second electrode assembly including:
(i) a stimulus electrode, and
(ii) a sensing electrode, the stimulus and sensing electrodes being positioned on opposing lateral sides in relation to the longitudinal axis of the shaft assembly; and
(d) a controller, the controller being operable to:
(i) generate an electrical signal to perform one or both of tissue ablation or denervation of a targeted nerve via the first electrode assembly,
(ii) generate an electrical stimulus signal to stimulate the targeted nerve via the stimulus electrode of the second electrode assembly, and
(iii) process a response signal received from the targeted nerve via the sensing electrode of the second electrode assembly.

2. The apparatus of claim 1, the shaft assembly having a rigid portion and a flexible portion, the flexible portion including the distal end.

3. The apparatus of claim 2, further comprising an actuator, the actuator being operable to deflect the flexible portion and thereby drive the distal end laterally relative to the longitudinal axis.

4. The apparatus of claim 1, further comprising a camera assembly at the distal end.

5. The apparatus of claim 1, further comprising a position sensor, the position sensor being operable to generate a signal indicating a position of the distal end in three-dimensional space.

6. The apparatus of claim 1, further comprising a cuff at the distal end, the first electrode assembly being secured to the distal end via the cuff.

7. The apparatus of claim 6, the second electrode assembly being secured to the distal end via the cuff.

8. The apparatus of claim 6, the cuff being formed of an electrically insulative material.

9. The apparatus of claim 1, the first electrode assembly including a first electrode and a second electrode.

10. The apparatus of claim 9, the first electrode and the second electrode cooperating to define a generally circular shape encircling the longitudinal axis.

11. The apparatus of claim 9, the first electrode and the second electrode being angularly spaced apart from each other by a first gap and a second gap.

12. The apparatus of claim 9, the first electrode and the second electrode each having an arcuate shape.

13. The apparatus of claim 9, the first electrode and the second electrode facing distally away from the distal end of the shaft assembly.

14. The apparatus of claim 1, the second electrode assembly further including:
(i) a first shaft, the stimulus electrode being secured to the first shaft, and
(ii) a second shaft, the sensing electrode being secured to the second shaft.

15. The apparatus of claim 1, further comprising an actuator, the actuator being operable to drive the second electrode assembly longitudinally relative to the shaft assembly.

16. The apparatus of claim 1, the stimulus electrode comprising a first needle electrode, the sensing electrode comprising a second needle electrode.

17. The apparatus of claim 1, the first electrode assembly including a first electrode and a second electrode, the stimulus electrode being angularly interposed between the first electrode and the second electrode.

18. The apparatus of claim 1, the controller being further operable to generate an electrical stimulus signal having a modulated waveform.

19. An apparatus, comprising:
(a) a shaft assembly having a distal end, the shaft assembly being configured to fit in a nasal cavity of a patient, the shaft assembly defining a longitudinal axis;
(b) a first electrode assembly at the distal end of the shaft assembly, the first electrode assembly including a distally-facing first electrode;
(c) a second electrode assembly at the distal end of the shaft assembly, the second electrode assembly including:
(i) a stimulus electrode, and
(ii) a sensing electrode; and
(d) a controller, the controller being operable to:
(i) generate an electrical signal to perform one or both of tissue ablation or denervation of a targeted nerve via the first electrode assembly,
(ii) generate an electrical stimulus signal to stimulate the targeted nerve via the stimulus electrode of the second electrode assembly, and
(iii) process a response signal received from the targeted nerve via the sensing electrode of the second electrode assembly.

20. A method comprising:
(a) inserting a shaft assembly into a nasal cavity of a patient, the shaft assembly defining a longitudinal axis;
(b) engaging tissue of the patient with a first electrode assembly;
(c) applying electrical energy to the tissue via the first electrode assembly;
(d) engaging the tissue with a second electrode assembly;
(e) applying an electrical stimulus to a nerve associated with the tissue, the electrical stimulus being applied via a stimulus electrode of the second electrode assembly, the stimulus electrode contacting the tissue at a first lateral side of the longitudinal axis; and
(f) determining whether a signal is received via a sensing electrode of the second electrode assembly in response to the electrical stimulus applied via the stimulus electrode, the sensing electrode contacting the tissue at a second lateral side of the longitudinal axis.
